(19) United States
(12) Patent Application Publication
(10) Pub. No.: US 2006/0011262 A1
(43) Pub. Date: Jan. 19, 2006

(54) Beverage bottling plant for filling bottles with a liquid beverage material having a treatment device for the treatment of bottles

(76) Inventor: Thomas Stienen, Unna (DE)

Correspondence Address: NILS H. LJUNGMAN & ASSOCIATES, P. O. BOX 130, GREENSBURG, PA, 15601-0130, US

(21) Appl. No.: 11/156,267

(22) Filed: Jun. 17, 2005

(30) Foreign Application Priority Data
Jun. 19, 2004 (DE) 10 2004 029 803.3

(51) Int. Cl. B65B 43/42

(52) U.S. Cl. 141/147

(57) ABSTRACT

A beverage bottling plant for filling bottles with a liquid beverage material having a treatment device for the treatment of bottles. The abstract of the disclosure is submitted herewith as required by 37 C.F.R. §1.72(b). As stated in 37 C.F.R. §1.72(b): A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims. Therefore, any statements made relating to the abstract are not intended to limit the claims in any manner and should not be interpreted as limiting the claims in any manner.

### BACKGROUND

1. Technical Field

The present application relates to a beverage bottling plant for filling bottles with a liquid beverage material having a treatment device for the treatment of bottles or containers.

2. Background Information

A beverage bottling plant for filling bottles with a liquid beverage filling material can possibly comprise a beverage filling machine with a plurality of beverage filling positions, each beverage filling position having a beverage filling device for filling bottles with liquid beverage filling material. The filling devices may have an apparatus designed to introduce a predetermined volume of liquid beverage filling material into the interior of bottles to a substantially predetermined level of liquid beverage filling material. The apparatus designed to introduce a predetermined flow of liquid beverage filling material further comprises an apparatus that is designed to terminate the filling of the beverage bottles upon the liquid beverage filling material reaching the predetermined level in bottles. There may also be provided a conveyer arrangement that is designed to move bottles, for example, from an inspecting machine to the filling machine. Upon filling, a closing station closes the filled bottles. There may further be provided a conveyer arrangement configured to transfer filled bottles from the filling machine to the closing station. Bottles may be labeled in a labeling station, the labeling station having a conveyer arrangement to receive bottles and to output bottles. The closing station and the labeling station may be connected by a corresponding conveyer arrangement.

In the beverage industry, for example, e.g. for the bottling of beverages in bottles or similar containers, it is frequently necessary to treatg these containers, at least on their interior surfaces, prior to bottling to achieve the required sterility and thus the shelf life of the bottled product. One process that is widely used is hydrogen peroxide treatment. In one of these methods, liquid hydrogen peroxide, for example, is finely atomized and mixed with a current of air, whereby the air is generally a current of sterile air. Then this hydrogen peroxide mixture is fed to a vaporizer in which any hydrogen peroxide that is still liquid is completely vaporized. Then this mixture of vapor and air is introduced into the containers to be sterilized, where the hydrogen peroxide immediately condenses on the cold interior walls of the container, where it forms a uniform liquid film. For the subsequent activation of the hydrogen peroxide, i.e. to initiate the decomposition of the hydrogen peroxide, it is necessary to heat it to a specified temperature, or to add a specified amount of heat to it, which as a rule is transferred to the hydrogen peroxide by means of an activation medium. In most cases, this activation medium is sterile warm air that is injected into the containers and has been heated to the temperature required for the activation of the hydrogen peroxide.

During the decomposition process, the hydrogen peroxide decomposes into water and free radicals, namely atomic oxygen 0 and HO groups which essentially perform the actual sterilization. After the conclusion of the sterilization, the containers are blown out by means of rinsing air and/or dry air and dried.

The prior art also reports methods in which hydrogen peroxide that is already in vapor form is activated by further temperature increases and caused to transition into the decomposition stage.

The methods of the prior art that work with temperature activation have numerous disadvantages.

In the first place, the long time required for the activation and decomposition of the hydrogen peroxide into its active components must be cited as a major disadvantage. On the systems that are realized in practice, cycle times for activation, sterilization and subsequent rinsing with rinsing air and/or dry air up to 20 seconds have been measured, which, given the high outputs desired by the customers who operate bottling machines etc., means that a bottling machine has to have two or even three bottle treatment machines or sterilization devices, which represents an excessively expensive solution.

With the temperature activation of the hydrogen peroxide, it is also a major disadvantage that the plastic bottles which are increasingly common are subject to a temperature limitation, as a result of which the maximum activation temperature is limited. On account of the decomposition period of the hydrogen peroxide, which becomes superproportionally shorter as the activation temperature increases, the highest possible activation temperature is desirable, although if this temperature exceeds the maximum temperature that can be withstood by the plastic bottles, there can be undesirable effects such as, for example, the shrinkage and/or deformation of the plastic bottles, which has adverse consequences on the cost of the bottling material.

An additional disadvantage of temperature activation is that, in particular when only the interior walls of the plastic surfaces are sterilized, more or less great temperature differences can occur in the walls of the plastic bottles, as a result of which, e.g. by the formation of cracks, the shelf life can be reduced and there can be adverse effects with regard to the optical appearance of the containers.

### OBJECT OR OBJECTS

The object is to reliably eliminate the disadvantages described above with the simultaneous achievement of a significantly shorter activation or decomposition time for the hydrogen peroxide. For this purpose, the present application teaches that the decomposition of the hydrogen peroxide is accelerated by the use of at least one catalyst, whereby the necessary decomposition temperature is likewise reduced.

The above-discussed embodiments of the present invention will be described further hereinbelow. When the word “invention” or “embodiment of the invention” is used in this specification, the word “invention” or “embodiment of the invention” includes “inventions” or “embodiments of the invention”, that is the plural of “invention” or “embodiment of the invention”. By stating “invention” or “embodiment of the invention”, the Applicant does not in any way admit that the present application does not include more than one patentably and non-obviously distinct invention, and maintains that this application may include more than one patentably and non-obviously distinct invention. The Applicant hereby asserts that the disclosure of this application may include more than one invention, and, in the event that there is more than one invention, that these inventions may be patentable and non-obvious one with respect to the other.

### BRIEF DESCRIPTION OF THE DRAWINGS

The present application is explained in greater detail below on the basis of an exemplary embodiment which is illustrated in the accompanying drawings, in which:

FIG. 1A is a schematic illustration of a container filling plant in accordance with one possible embodiment;

FIG. 1A is a schematic illustration of a container filling plant in accordance with one possible embodiment;

FIGS. 1 and 3 are simplified sectional views of sterilizer heads;

FIG. 2 is a simplified overhead view of a bottle treatment machine that is realized in the form of a rotary machine;

FIG. 3A is similar to FIG. 3 and shows further detail of a sterilizer head;

FIG. 4 is a close-up view of a tube in a bottle treatment machine with an enhancer installed therein.

Developments, advantages and potential applications of the present application are indicated in the following description of exemplary embodiments and of the drawing. All the features described and or illustrated are the object of the present application, individually or in any possible combination, regardless of their placement in the claims or the references between claims. The text of the claims is also incorporated by reference into the description.

### DESCRIPTION OF EMBODIMENT OR EMBODIMENTS

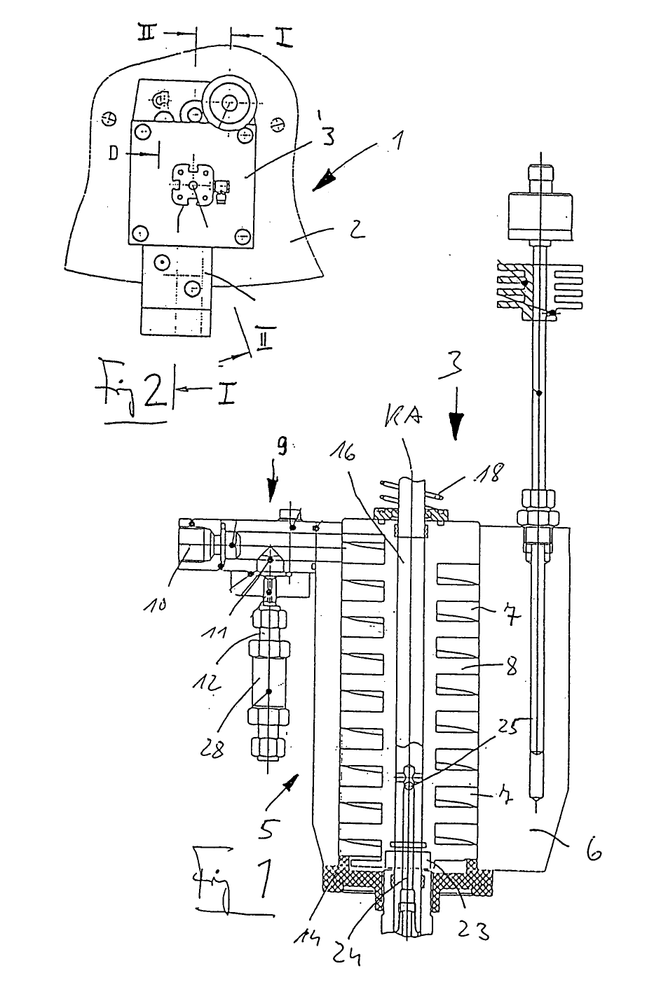

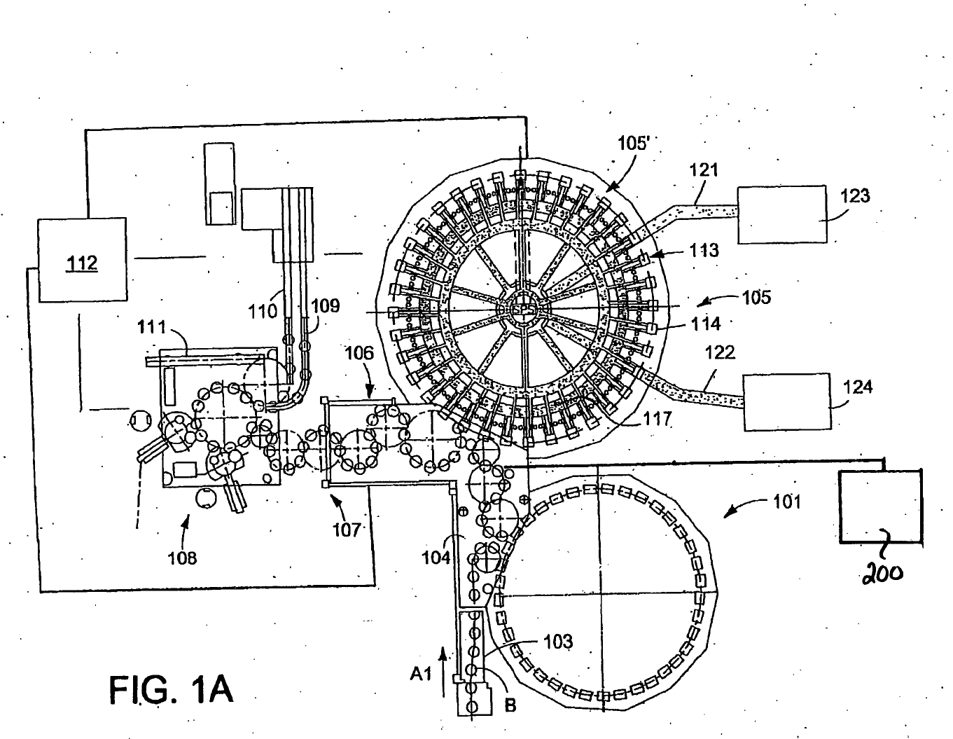

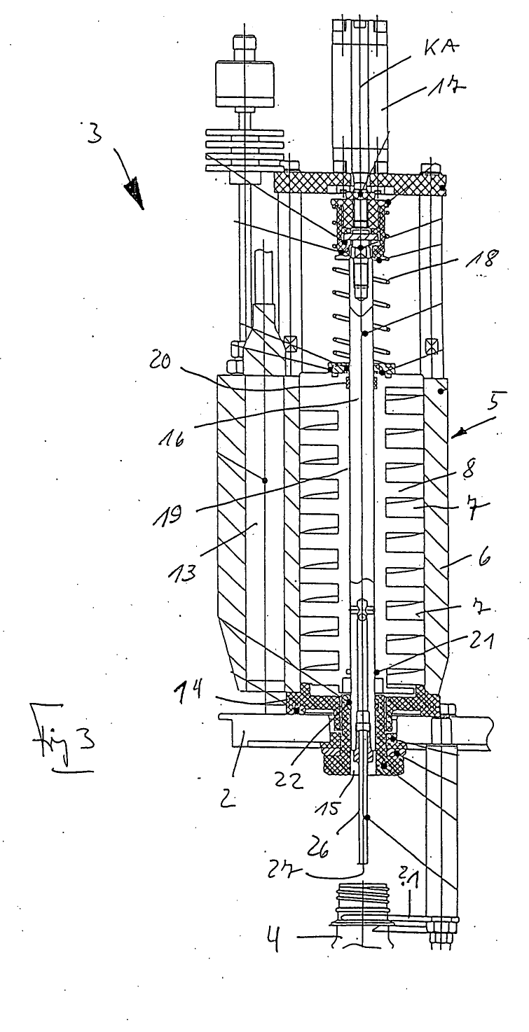

FIG. 1A shows schematically the main components of one possible embodiment example of a system for filling containers, specifically, a beverage bottling plant for filling bottles B with at least one liquid beverage, in accordance with at least one possible embodiment, in which system or plant could possibly be utilized at least one aspect, or several aspects, of the embodiments disclosed herein.

FIG. 1A shows a rinsing arrangement or rinsing station 101, to which the containers, namely bottles B, are fed in the direction of travel as indicated by the arrow A1, by a first conveyer arrangement 103, which can be a linear conveyor or a combination of a linear conveyor and a starwheel. Downstream of the rinsing arrangement or rinsing station 101, in the direction of travel as indicated by the arrow A1, the rinsed bottles B are transported to a beverage filling machine 105 by a second conveyer arrangement 104 that is formed, for example, by one or more starwheels that introduce bottles B into the beverage filling machine 105.

The beverage filling machine 105 shown is of a revolving or rotary design, with a rotor 105′, which revolves around a central, vertical machine axis. The rotor 105′ is designed to receive and hold the bottles B for filling at a plurality of filling positions 113 located about the periphery of the rotor 105′. At each of the filling positions 103 is located a filling arrangement 114 having at least one filling device, element, apparatus, or valve. The filling arrangements 114 are designed to introduce a predetermined volume or amount of liquid beverage into the interior of the bottles B to a predetermined or desired level.

The filling arrangements 114 receive the liquid beverage material from a toroidal or annular vessel 117, in which a supply of liquid beverage material is stored under pressure by a gas. The toroidal vessel 117 is a component, for example, of the revolving rotor 105′. The toroidal vessel 117 can be connected by means of a rotary coupling or a coupling that permits rotation. The toroidal vessel 117 is also connected to at least one external reservoir or supply of liquid beverage material by a conduit or supply line. In the embodiment shown in FIG. 1A, there are two external supply reservoirs 123 and 124, each of which is configured to store either the same liquid beverage product or different products. These reservoirs 123, 124 are connected to the toroidal or annular vessel 117 by corresponding supply lines, conduits, or arrangements 121 and 122. The external supply reservoirs 123, 124 could be in the form of simple storage tanks, or in the form of liquid beverage product mixers, in at least one possible embodiment.

As well as the more typical filling machines having one toroidal vessel, it is possible that in at least one possible embodiment there could be a second toroidal or annular vessel which contains a second product. In this case, each filling arrangement 114 could be connected by separate connections to each of the two toroidal vessels and have two individually-controllable fluid or control valves, so that in each bottle B, the first product or the second product can be filled by means of an appropriate control of the filling product or fluid valves.

Downstream of the beverage filling machine 105, in the direction of travel of the bottles B, there can be a beverage bottle closing arrangement or closing station 106 which closes or caps the bottles B. The beverage bottle closing arrangement or closing station 106 can be connected by a third conveyer arrangement 107 to a beverage bottle labeling arrangement or labeling station 108. The third conveyor arrangement may be formed, for example, by a plurality of starwheels, or may also include a linear conveyor device.

In the illustrated embodiment, the beverage bottle labeling arrangement or labeling station 108 has at least one labeling unit, device, or module, for applying labels to bottles B. In the embodiment shown, the labeling arrangement 108 has three output conveyer arrangement: a first output conveyer arrangement 109, a second output conveyer arrangement 110, and a third output conveyer arrangement 111, all of which convey filled, closed, and labeled bottles B to different locations.

The first output conveyer arrangement 109, in the embodiment shown, is designed to convey bottles B that are filled with a first type of liquid beverage supplied by, for example, the supply reservoir 123. The second output conveyer arrangement 110, in the embodiment shown, is designed to convey bottles B that are filled with a second type of liquid beverage supplied by, for example, the supply reservoir 124. The third output conveyer arrangement 111, in the embodiment shown, is designed to convey incorrectly labeled bottles B. To further explain, the labeling arrangement 108 can comprise at least one beverage bottle inspection or monitoring device that inspects or monitors the location of labels on the bottles B to determine if the labels have been correctly placed or aligned on the bottles B. The third output conveyer arrangement 111 removes any bottles B which have been incorrectly labeled as determined by the inspecting device.

The beverage bottling plant can be controlled by a central control arrangement 112, which could be, for example, computerized control system that monitors and controls the operation of the various stations and mechanisms of the beverage bottling plant.

FIG. 1A further shows a treatment or sterilization station, represented by a box 200, disposed between the bottle cleaning machine 101 and the bottle filling machine 105.

FIG. 1 is a simplified illustration of a sterilizer head 3, as it can be located on a plurality of machines, both on rotary machines and on linear machines, for the disinfection of containers.

The sterilizer head 3 which serves as the exemplary embodiment is used for the hydrogen peroxide sterilization of containers, e.g. temperature-sensitive PET bottles.

Provided underneath each of the sterilizer heads 3, which are located, for example, at uniform angular intervals on the periphery of the rotor 2, is a container or bottle carrier, in which the individual bottle to be sterilized is suspended, for example, and namely so that it is equi-axial with a vertical sterilizer head axis KA, so that when the sterilizer head 3 is activated, a stream of hot sterilization medium can be dispensed into the interior of the bottle 4 to be sterilized, which is located underneath the sterilization head 3.

The sterilizer head 3 comprises a heater or heat exchanger 5 with a housing 6 in which, among other things, a flow channel or heating channel 7 is realized that surrounds the axis KA in a helical fashion, and namely in the illustrated embodiment so that a core 8 is inserted in a cylindrical opening of the housing 6, which opening is coaxial with the axis KA, and which core has a helical groove on its periphery that forms the flow channel 7.

The upper end of the flow channel 7 shown in FIG. 1, which is closed toward the outside, is in communication with a sprayer device 9 which supplies sterile compressed air via the connection 10 and which has a spray nozzle 11 which is supplied with hydrogen peroxide by means of the connection 12 and a pump (not shown), e.g. a membrane pump, which is connected with this connection, whereby the hydrogen peroxide is then introduced to form the hydrogen peroxide-air aerosol via the spray nozzle 11 in the form of a finely sprayed jet or mist into the air flow that is flowing through the spray device 9, so that this aerosol which comprises air and hydrogen peroxide then arrives in the flow channel 7 of the heat exchanger 5.

By means of a heating device (not shown), which can be formed, for example, by an electrical heating cartridge with a thermosensor, the heat exchanger 5 and in particular the core 8 are heated.

The lower end of the housing 6 is formed by suitable elements which make it possible to dispense the vapor-air mixture containing the hydrogen peroxide continuously or discontinuously, directly or indirectly, into the bottles 4 to be sterilized. For that purpose a tube 26 can be used, for example, which can extend with its dispensing opening 27 at least partly into the bottles 4, or can also be positioned with its dispensing opening 27 directly in front of the mouth of the bottles 4.

The heat exchanger is heated by the heater device, and namely to a temperature that is determined taking into consideration all the relevant parameters such as, for example, the volume and/or mass flow of the aerosol containing the hydrogen peroxide, the initial temperature of the aerosol, the specific thermal capacity etc., so that the hydrogen peroxide contained in the aerosol is vaporized to the desired extent not later than when it leaves the heat exchanger 5.

In similar devices of the prior art, the conventional practice is to heat the heat exchanger 5, for example, to temperatures on the order of magnitude of 130-150° C., as a result of which additional measures are frequently necessary to prevent an overheating of the bottles 4. The present application teaches that inside the path of the hydrogen peroxide or the path of the aerosol that contains the hydrogen peroxide there is at least one catalyst to initiate and assist the decomposition process of the hydrogen peroxide at low temperatures.

As a result of the presence of the at least one catalyst taught by the present application, the activation energy required for the initiation of the decomposition process of the hydrogen peroxide is reduced, so that the decomposition proceeds rapidly even at room temperature, for example, as a result of which the effective free radicals do not have to be produced first by time-consuming thermal activation inside the bottles 4, but can be introduced directly into the bottles.

As the chemically active material for the at least one catalyst, all the materials known to the prior art and suitable for this application can be used, such as, for example, the particularly suitable noble metals of the platinum group, but also, for example, magnesium dioxide or brownstone and other substances.

It is therefore appropriate to use these materials in a form that has a particularly large active surface. In particular the use of wire meshes manufactured from small-diameter wire is preferred, and/or the use of catalyst elements manufactured using powder-metallurgical processes.

Several possibilities for the at least one catalyst are listed below by way of example, and without any intent to restrict the scope of the present application.

The present application teaches that the interior of the tube 26 is filled at least partly with a catalyst.

The present application also teaches that a catalyst which comprises preferably porous material is realized in the form of a dispensing element for the decomposing or already decomposed hydrogen peroxide, whereby the exterior surface of this dispensing element is realized airtight. The mouth of this dispending catalyst element ends in front of the mouth of the container 4 and/or inside the container 4.

The present application also teaches that the surface of the heat exchanger 5 is coated at least partly with a material that acts as a catalyst, or the heat exchanger 5 is manufactured at least partly from such a material.

The present application further teaches that at least one catalyst is directly associated with each sterilizer head or each treatment station, and/or at least one catalyst is associated with a group of sterilizer heads, and/or at least one catalyst is associated with all the sterilizer heads of a sterilization machine in common.

In an additional configuration of the present application, the at least one catalyst is realized in the form of an (additional) heating element, as a result of which there are additional possibilities for the targeted control of the decomposition of the hydrogen peroxide.

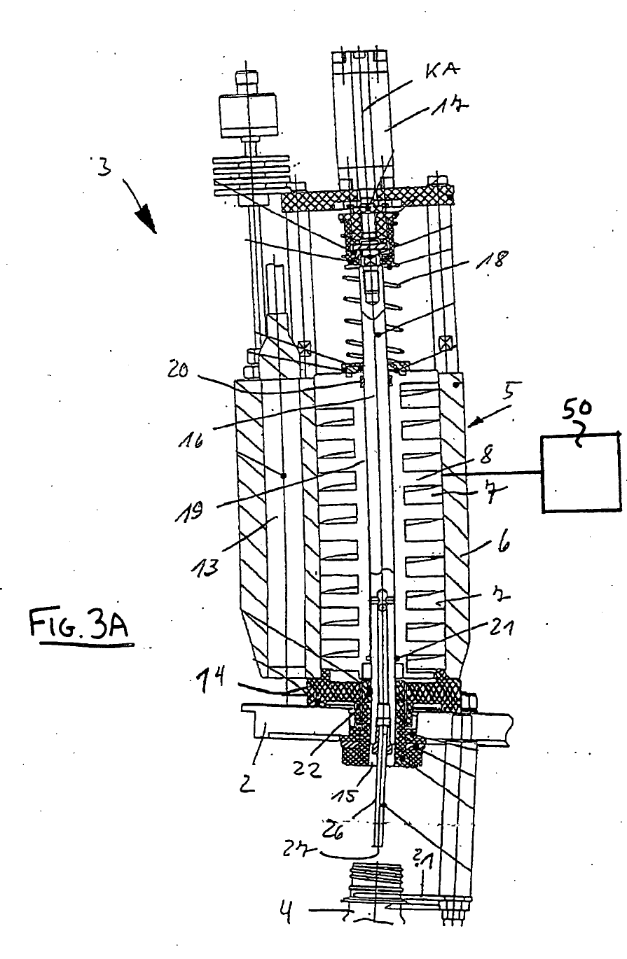

FIG. 3A shows an additional heating element 50 for the targeted control of the decomposition of the hydrogen peroxide, according to another possible embodiment.

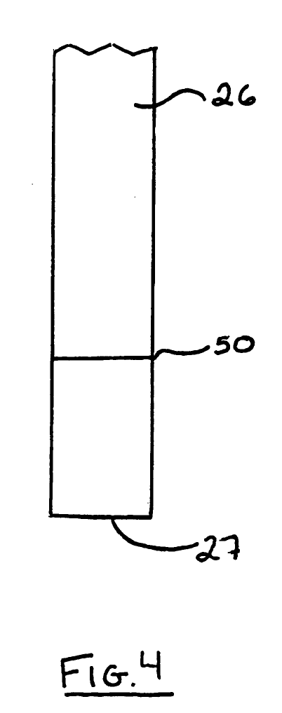

FIG. 4 shows a close-up view of an embodiment of the tube 26 with its dispensing end 27, and an enhancer device 50 installed therein. In one possible embodiment, the enhancer device 50 could comprise a thin wire mesh material, and could be disposed near the end of the tube 26. However, the enhancer device 50 could be disposed at any point in the tube 26, closer to the dispensing end 27 or farther away from the dispensing end 27 in other possible embodiments. Further, the enhancer device could take any of the above-mentioned forms in the treatment machine in other possible embodiments.

The present application relates to a container treatment machine, in particular for the sterilization of bottles made of plastic, metal or glass, cans or similar containers, by means of hydrogen peroxide, realized in the form of a rotary or linear machine, whereby at least one catalyst that promotes the decomposition of the hydrogen peroxide is located in the flow path of the liquid and/or gaseous hydrogen peroxide.

An example of a sterilization machine, components of which may possibly be utilized or adapted for use in at least one possible embodiment, may possibly be found in the Innosept machine manufactured by Applicant, a description of which may be found on Applicant's website, www.khs-ag.com. Further information regarding sterilization devices and methods produced by Applicant which may be utilized or adapted for use in at least one possible embodiment may be found in an article entitled “Coming In to the Cold Fill: New “Alfill” Technology from KHS Makes Aseptic Cold-Fill Simple, Economical, Environmentally Sound, and Flexible,” written by Jörn Fehland, who is manager of KHS Alfill, Center of Competence Aseptic, Hamburg, Germany, which article may be found at the following website: http://www.asepticinfo.com/issues/2004/sep/3.aspx.

One feature or aspect of an embodiment is believed at the time of the filing of this patent application to possibly reside broadly in a container treatment machine, in particular for the sterilization of bottles made of plastic, metal or glass, cans or similar containers, by means of hydrogen peroxide, realized in the form of a rotary or linear machine, characterized by the fact that at least one catalyst that promotes the decomposition of the hydrogen peroxide is located in the flow path of the liquid and/or gaseous hydrogen peroxide.

Another feature or aspect of an embodiment is believed at the time of the filing of this patent application to possibly reside broadly in a container treatment machine, characterized by the fact that the at least one catalyst is located on the path of the hydrogen peroxide from its source to the container to be treated.

Yet another feature or aspect of an embodiment is believed at the time of the filing of this patent application to possibly reside broadly in a Container treatment, characterized by the fact that the at least one catalyst is located inside a tube that conducts the hydrogen peroxide and/or its decomposition products into or to the container.

Still another feature or aspect of an embodiment is believed at the time of the filing of this patent application to possibly reside broadly in a container treatment machine, characterized by the fact that the catalyst is also realized in the form of a tube that conducts the hydrogen peroxide and/or its decomposition products in or to the container and is made of a porous material.

A further feature or aspect of an embodiment is believed at the time of the filing of this patent application to possibly reside broadly in a container treatment machine as claimed in one of the preceding claims, characterized by the fact that the surface of the heat exchanger is at least partly realized in the form of a catalyst.

Another feature or aspect of an embodiment is believed at the time of the filing of this patent application to possibly reside broadly in a container treatment machine, characterized by the fact that the heat exchanger is made at least partly of catalytically active material.

Yet another feature or aspect of an embodiment is believed at the time of the filing of this patent application to possibly reside broadly in a container treatment machine, characterized by the fact that the at least one catalyst is made of noble metals of the platinum group and/or magnesium dioxide or brownstone.

Still another feature or aspect of an embodiment is believed at the time of the filing of this patent application to possibly reside broadly in a as claimed in one of the preceding claims, characterized by the fact that the at least one catalyst is manufactured by means of powder-metallurgical processes.

A further feature or aspect of an embodiment is believed at the time of the filing of this patent application to possibly reside broadly in a container treatment machine, characterized by the fact that the at least one catalyst comprises wire meshes manufactured from small-diameter wire.

Another feature or aspect of an embodiment is believed at the time of the filing of this patent application to possibly reside broadly in a container treatment machine, characterized by the fact that the at least one catalyst is realized in the form of a heating element for the hydrogen peroxide and/or its decomposition products.

Yet another feature or aspect of an embodiment is believed at the time of the filing of this patent application to possibly reside broadly in a container treatment machine, characterized by the fact that at least one catalyst is associated directly with each sterilizer.

Still another feature or aspect of an embodiment is believed at the time of the filing of this patent application to possibly reside broadly in a container treatment machine, characterized by the fact that at least one catalyst is directly associated with a group of sterilizers.

A further feature or aspect of an embodiment is believed at the time of the filing of this patent application to possibly reside broadly in a container treatment machine, characterized by the fact that at least one catalyst is directly associated with all the sterilizers of a container treatment machine.

Another feature or aspect of an embodiment is believed at the time of the filing of this patent application to possibly reside broadly in a method for the operation of a container treatment machine, characterized by the fact that the decomposition of the hydrogen peroxide takes place at a temperature below the maximum allowable temperature of the containers to be treated.

Yet another feature or aspect of an embodiment is believed at the time of the filing of this patent application to possibly reside broadly in a method for the operation of a container treatment machine, characterized by the fact that the decomposition of the hydrogen peroxide takes place at a temperature below the shape recovery and/or contraction temperature of plastic containers.

Still another feature or aspect of an embodiment is believed at the time of the filing of this patent application to possibly reside broadly in a method for the operation of a container treatment machine, characterized by the fact that the decomposition of the hydrogen peroxide occurs at room temperature, preferably in the temperature range of 15 to 25° C.

A further feature or aspect of an embodiment is believed at the time of the filing of this patent application to possibly reside broadly in a beverage bottling plant for filling beverage bottles with liquid beverage material, said beverage bottling plant comprising: a beverage bottle cleaning machine being configured and disposed to clean beverage bottles; a feed arrangement to supply beverage bottles to said beverage bottle cleaning machine; a beverage filling machine being configured and disposed to fill beverage bottles with liquid beverage material; said beverage filling machine comprising a plurality of beverage filling elements for filling beverage bottles with liquid beverage material; at least one liquid reservoir being configured to hold a liquid to be bottled; said at least one liquid reservoir comprising a gas headspace being disposed above a liquid to be bottled within said at least one liquid reservoir; at least one supply line being configured and disposed to connect said at least one liquid reservoir to said beverage filling machine to supply liquid beverage material to said beverage filling machine; a first conveyer arrangement being configured and disposed to move beverage bottles from said beverage bottle cleaning machine into said beverage filling machine; said first conveyer arrangement comprising a star wheel structure; a beverage bottle closing machine being configured and disposed to close tops of filled beverage bottles; a second conveyer arrangement being configured and disposed to move filled beverage bottles from said beverage filling machine into said beverage bottle closing machine; said second conveyer arrangement comprising a star wheel structure; a beverage bottle labeling machine being configured and disposed to label filled, closed beverage bottles; a third conveyor arrangement being configured and disposed to move filled, closed beverage bottles from said beverage bottle closing machine into said beverage bottle labeling machine; said third conveyer arrangement comprising a star wheel structure; a beverage bottle packing station being configured and disposed to package labeled, filled, closed beverage bottles; a fourth conveyor arrangement being configured and disposed to move labeled, filled, closed beverage bottles from said beverage bottle labeling machine to said beverage bottle packing station; said fourth conveyer arrangement comprising a linear conveyor structure being configured and disposed to arrange beverage bottles in groups for packing; a computer control system being configured and disposed to monitor and control operation of said beverage bottling plant; a beverage bottle treatment machine being configured and disposed to treat bottles to be filled; said beverage bottle treatment machine comprising: a treatment device being configured and disposed to treat the insides of bottles to be filled, comprising: a supply for containing treatment agent to treat bottles; a spray device being configured and disposed to introduce compressed air into said treatment device and to mix said compressed air with a supply of treatment agent to create treated air to treat bottles; a heater or heat exchanger being configured and disposed to heat treated air; a helical heating channel being configured and disposed to heat treated air and to permit the flow of treated air; a vertical tube being configured and disposed to permit the flow of treated air into a bottle; said tube comprising a dispensing opening being configured and disposed to permit the flow of treated air into a bottle; said helical heating channel being disposed about said tube; a housing being configured and disposed to house said heater or heat exchanger, said tube, and said helical heating channel; an enhancer being configured and disposed to enhance treated air prior to the exit of treated air from said tube.

Another feature or aspect of an embodiment is believed at the time of the filing of this patent application to possibly reside broadly in a method of treating bottles with a treatment device, said treatment device comprising: a supply for containing treatment agent to treat bottles; a spray device being configured and disposed to introduce compressed air into said treatment device and to mix said compressed air with a supply of treatment agent to create treated air to treat bottles; a heater or heat exchanger being configured and disposed to heat treated air; a helical heating channel being configured and disposed to heat treated air and to permit the flow of treated air; a vertical tube being configured and disposed to permit the flow of treated air into a bottle; said tube comprising a dispensing opening being configured and disposed to permit the flow of treated air into a bottle; said helical heating channel being disposed about said tube; a housing being configured and disposed to house said heater or heat exchanger, said tube, and said helical heating channel; an enhancer being configured and disposed to enhance treated air prior to the exit of treated air from said tube; said method comprising the steps of: mixing treatment agent and compressed air to create treated air; heating treated air with said heater or heat exchanger; flowing air through said enhancer to enhance treated air; enhancing treated air at a temperature below the maximum allowable temperature of the containers to be treated; flowing enhanced, treated air into a container to be treated.

Yet another feature or aspect of an embodiment is believed at the time of the filing of this patent application to possibly reside broadly in a container treatment device being configured and disposed to treat the insides of containers to be filled, comprising: a supply for containing treatment agent to treat containers; a spray device being configured and disposed to introduce compressed air into said treatment device and to mix said compressed air with a supply of treatment agent to create a treated air to treat containers; a heater or heat exchanger being configured and disposed to heat treated air; a vertical tube being configured and disposed to permit the flow of treated air into a container; said tube comprising a dispensing opening being configured and disposed to permit the flow of treated air into a container; an enhancer being configured and disposed to enhance treated air exiting from said tube.

The components disclosed in the various publications, disclosed or incorporated by reference herein, may possibly be used in possible embodiments of the present invention, as well as equivalents thereof.

Some examples of bottling systems, which may be used or adapted for use in at least one possible embodiment of the present may be found in the following U.S. Patents assigned to the Assignee herein, namely: U.S. Pat. No. 4,911,285; No. 4,944,830; No. 4,950,350; No. 4,976,803; No. 4,981,547; No. 5,004,518; No. 5,017,261; No. 5,062,917; No. 5,062,918; No. 5,075,123; No. 5,078,826; No. 5,087,317; No. 5,110,402; No. 5,129,984; No. 5,167,755; No. 5,174,851; No. 5,185,053; No. 5,217,538; No. 5,227,005; No. 5,413,153; No. 5,558,138; No. 5,634,500; No. 5,713,403; No. 6,276,113; No. 6,213,169; No. 6,189,578; No. 6,192,946; No. 6,374,575; No. 6,365,054; No. 6,619,016; No. 6,474,368; No. 6,494,238; No. 6,470,922; and No. 6,463,964.

The purpose of the statements about the technical field is generally to enable the Patent and Trademark Office and the public to determine quickly, from a cursory inspection, the nature of this patent application. The description of the technical field is believed, at the time of the filing of this patent application, to adequately describe the technical field of this patent application. However, the description of the technical field may not be completely applicable to the claims as originally filed in this patent application, as amended during prosecution of this patent application, and as ultimately allowed in any patent issuing from this patent application. Therefore, any statements made relating to the technical field are not intended to limit the claims in any manner and should not be interpreted as limiting the claims in any manner.

Some examples of control systems which measure operating parameters and learn therefrom that may possibly be utilized or possibly adapted for use in at least one possible embodiment of the present application may possibly be found in the following U.S. patents: U.S. Pat. No. 4,655,188 issued to Tomisawa et al. on Apr. 7, 1987; U.S. Pat. No. 5,191,272 issued to Torii et al. on Mar. 2, 1993; U.S. Pat. No. 5,223,820, issued to Sutterlin et al. on Jun. 29, 1993; and U.S. Pat. No. 5,770,934 issued to Theile on Jun. 23, 1998.

The appended drawings in their entirety, including all dimensions, proportions and/or shapes in at least one embodiment of the invention, are accurate and are hereby included by reference into this specification.

Some examples of stepping motors that may possibly be utilized or possibly adapted for use in at least one possible embodiment of the present application may possibly be found in the following U.S. patents: U.S. Pat. No. 6,348,774 issued to Andersen et al. on Feb. 19, 2002; U.S. Pat. No. 6,373,209 issued to Gerber et al. on Apr. 16, 2002; U.S. Pat. No. 6,424,061 issued to Fukuda et al. on Jul. 23, 2002; U.S. Pat. No. 6,509,663 issued to Aoun on Jan. 21, 2003; U.S. Pat. No. 6,548,923 to Ohnishi et al. on Apr. 15, 2003; and U.S. Pat. No. 6,661,193 issued to Tsai on Dec. 9, 2003.

The background information is believed, at the time of the filing of this patent application, to adequately provide background information for this patent application. However, the background information may not be completely applicable to the claims as originally filed in this patent application, as amended during prosecution of this patent application, and as ultimately allowed in any patent issuing from this patent application. Therefore, any statements made relating to the background information are not intended to limit the claims in any manner and should not be interpreted as limiting the claims in any manner.

Some examples of servo-motors that may possibly be utilized or possibly adapted for use in at least one possible embodiment of the present application may possibly be found in the following U.S. patents: U.S. Pat. No. 4,050,434 issued to Zbikowski et al. on Sep. 27, 1977; U.S. Pat. No. 4,365,538 issued to Andoh on Dec. 28, 1982; U.S. Pat. No. 4,550,626 issued to Brouter on Nov. 5, 1985; U.S. Pat. No. 4,760,699 issued to Jacobsen et al. on Aug. 2, 1988; U.S. Pat. No. 5,076,568 issued to de Jong et al. on Dec. 31, 1991; and No. 6,025 issued to Yasui on Feb. 15, 2000.

All, or substantially all, of the components and methods of the various embodiments may be used with at least one embodiment or all of the embodiments, if more than one embodiment is described herein.

Some examples of synchronous motors which may possibly be utilized or adapted for use in at least one possible embodiment may possibly be found in the following U.S. Patents: U.S. Pat. No. 6,713,899, entitled “Linear synchronous motor;” U.S. Pat. No. 6,486,581, entitled “Interior permanent magnet synchronous motor;” U.S. Pat. No. 6,424,114, entitled “Synchronous motor;” U.S. Pat. No. 6,388,353, entitled “Elongated permanent magnet synchronous motor;” U.S. Pat. No. 6,329,728, entitled “Cylinder-type linear synchronous motor;” U.S. Pat. No. 6,025,659, entitled “Synchronous motor with movable part having permanent magnets;” U.S. Pat. No. 5,936,322, entitled “Permanent magnet type synchronous motor;” and U.S. Pat. No. 5,448,123, entitled “Electric synchronous motor.”

The purpose of the statements about the object or objects is generally to enable the Patent and Trademark Office and the public to determine quickly, from a cursory inspection, the nature of this patent application. The description of the object or objects is believed, at the time of the filing of this patent application, to adequately describe the object or objects of this patent application. However, the description of the object or objects may not be completely applicable to the claims as originally filed in this patent application, as amended during prosecution of this patent application, and as ultimately allowed in any patent issuing from this patent application. Therefore, any statements made relating to the object or objects are not intended to limit the claims in any manner and should not be interpreted as limiting the claims in any manner.

Some examples of computer systems that may possibly be utilized or possibly adapted for use in at least one possible embodiment of the present application may possibly be found in the following U.S. Patents: U.S. Pat. No. 5,416,480 issued to Roach et al. on May 16, 1995; U.S. Pat. No. 5,479,355 issued to Hyduke on Dec. 26, 1995; U.S. Pat. No. 5,481,730 issued to Brown et al. on Jan. 2, 1996; U.S. Pat. No. 5,805,094 issued to Roach et al. on Sep. 8, 1998; U.S. Pat. No. 5,881,227 issued to Atkinson et al. on Mar. 9, 1999; and U.S. Pat. No. 6,072,462 issued to Moshovich on Jun. 6, 2000.

All of the patents, patent applications and publications recited herein, and in the Declaration attached hereto, are hereby incorporated by reference as if set forth in their entirety herein.

Some examples of pneumatic arrangements that may possibly be utilized or possibly adapted for use in at least one possible embodiment of the present application may possibly be found in the following U.S. Patents: U.S. Pat. No. 6,609,767 issued to Mortenson et al. on Aug. 26, 2003; U.S. Pat. No. 6,632,072 issued to Lipscomb et al. on Oct. 14, 2003; U.S. Pat. No. 6,637,838 issued to Watanabe on Oct. 28, 2003; U.S. Pat. No. 6,659,693 issued to Perkins et al. on Dec. 9, 2003; U.S. Pat. No. 6,668,848 issued to Ladler et al. on Dec. 30, 2003; and U.S. Pat. No. 6,676,229 issued to Marra et al. on Jan. 13, 2004.

The summary is believed, at the time of the filing of this patent application, to adequately summarize this patent application. However, portions or all of the information contained in the summary may not be completely applicable to the claims as originally filed in this patent application, as amended during prosecution of this patent application, and as ultimately allowed in any patent issuing from this patent application. Therefore, any statements made relating to the summary are not intended to limit the claims in any manner and should not be interpreted as limiting the claims in any manner.

Some examples of sterilizing or cleaning agents and concentrations thereof that may possibly be utilized or possibly adapted for use in at least one possible embodiment of the present application may possibly be found in the following U.S. Patents: U.S. Pat. No. 6,039,922 issued to Swank et al. on Mar. 21, 2000; U.S. Pat. No. 6,244,275 issued to Ziegler et al. on Jun. 12, 2001; U.S. Pat. No. 6,406,666 issued to Cicla et al. on Jun. 18, 2002; and U.S. Pat. No. 6,612,149 issued to Wang et al. on Sep. 2, 2003.

It will be understood that the examples of patents, published patent applications, and other documents which are included in this application and which are referred to in paragraphs which state “Some examples of . . . which may possibly be used in at least one possible embodiment of the present application . . . ” may possibly not be used or useable in any one or more embodiments of the application.

Some examples of heater arrangements that may possibly be utilized or possibly adapted for use in at least one possible embodiment of the present application may possibly be found in the following U.S. Patents: U.S. Pat. No. 6,404,421 issued to Meijler et al. on Jun. 11, 2002; U.S. Pat. No. 6,515,264 issued to Toya et al. on Feb. 4, 2003; U.S. Pat. No. 6,548,786 issued to Takizawa et al. on Apr. 15, 2003; U.S. Pat. No. 6,555,796 issued to Cusack on Apr. 29, 2003; U.S. Pat. No. 6,633,727 issued to Henie et al. on Oct. 14, 2003; and U.S. Pat. No. 6,677,557 issued to Ito et al. on Jan. 13, 2004.

The sentence immediately above relates to patents, published patent applications and other documents either incorporated by reference or not incorporated by reference.

Some examples of bottling systems which may possibly be utilized or adapted for use in at least one possible embodiment may possibly be found in the following U.S. patents: U.S. Pat. No. 6,684,602, entitled “Compact bottling machine;” U.S. Pat. No. 6,470,922, entitled “Bottling plant for bottling carbonated beverages;” U.S. Pat. No. 6,390,150, entitled “Drive for bottling machine;” U.S. Pat. No. 6,374,575, entitled “Bottling plant and method of operating a bottling plant;” U.S. Pat. No. 6,192,946, entitled “Bottling system;” U.S. Pat. No. 6,185,910, entitled “Method and an apparatus for high-purity bottling of beverages;” U.S. Pat. No. 6,058,985, entitled “Bottling machine with a set-up table and a set-up table for a bottling machine and a set-up table for a bottle handling machine;” U.S. Pat. No. 5,996,322, entitled “In-line bottling plant;” U.S. Pat. No. 5,896,899, entitled “Method and an apparatus for sterile bottling of beverages;” U.S. Pat. No. 5,848,515, entitled “Continuous-cycle sterile bottling plant;” U.S. Pat. No. 5,634,500, entitled “Method for bottling a liquid in bottles or similar containers;” and U.S. Pat. No. 5,425,402, entitled “Bottling system with mass filling and capping arrays.”

The corresponding foreign applications, namely, Federal Republic of Germany Patent Application No. 10 2004 029 803.3, filed on Jun. 19, 2004, having inventor Thomas Stienen, and DE-OS 10 2004 029 803.3 and DE-PS 10 2004 029 803.3, are hereby incorporated by reference as if set forth in their entirety herein for the purpose of correcting and explaining any possible misinterpretations of the English translation thereof. In addition, the published equivalents of the above corresponding foreign and international patent publication applications, and other equivalents or corresponding applications, if any, in corresponding cases in the Federal Republic of Germany and elsewhere, and the references and documents cited in any of the documents cited herein, such as the patents, patent applications and publications, are hereby incorporated by reference as if set forth in their entirety herein.

Some examples of starwheels which may possibly be utilized or adapted for use in at least one possible embodiment may possibly be found in the following U.S. Patents: U.S. Pat. No. 5,613,593, entitled “Container handling starwheel;” U.S. Pat. No. 5,029,695, entitled “Improved starwheel;” U.S. Pat. No. 4,124,112, entitled “Odd-shaped container indexing starwheel;” and U.S. Pat. No. 4,084,686, entitled “Starwheel control in a system for conveying containers.”

All of the references and documents, cited in any of the documents cited herein, are hereby incorporated by reference as if set forth in their entirety herein. All of the documents cited herein, referred to in the immediately preceding sentence, include all of the patents, patent applications and publications cited anywhere in the present application.

Some examples of heat exchangers which may possibly be utilized or adapted for use in at least one possible embodiment may possibly be found in the following U.S. Patents: U.S. Pat. No. 4,665,975, entitled “Plate type heat exchanger;” U.S. Pat. No. 6,810,948, entitled “Heat exchanger;” U.S. Pat. No. 6,799,428, entitled “Heat exchanger;” U.S. Pat. No. 6,394,179, entitled “Plate heat exchanger;” U.S. Pat. No. 6,125,649, entitled “Heat exchanger unit with conductive discs;” U.S. Pat. No. 5,579,650, entitled “Heat exchanger;” and U.S. Pat. No. 4,313,491, entitled “Coiled heat exchanger.”

The description of the embodiment or embodiments is believed, at the time of the filing of this patent application, to adequately describe the embodiment or embodiments of this patent application. However, portions of the description of the embodiment or embodiments may not be completely applicable to the claims as originally filed in this patent application, as amended during prosecution of this patent application, and as ultimately allowed in any patent issuing from this patent application. Therefore, any statements made relating to the embodiment or embodiments are not intended to limit the claims in any manner and should not be interpreted as limiting the claims in any manner.

The details in the patents, patent applications and publications may be considered to be incorporable, at applicant's option, into the claims during prosecution as further limitations in the claims to patentably distinguish any amended claims from any applied prior art.

The purpose of the title of this patent application is generally to enable the Patent and Trademark Office and the public to determine quickly, from a cursory inspection, the nature of this patent application. The title is believed, at the time of the filing of this patent application, to adequately reflect the general nature of this patent application. However, the title may not be completely applicable to the technical field, the object or objects, the summary, the description of the embodiment or embodiments, and the claims as originally filed in this patent application, as amended during prosecution of this patent application, and as ultimately allowed in any patent issuing from this patent application. Therefore, the title is not intended to limit the claims in any manner and should not be interpreted as limiting the claims in any manner.

The abstract of the disclosure is submitted herewith as required by 37 C.F.R. §1.72(b). As stated in 37 C.F.R. §1.72(b):

A brief abstract of the technical disclosure in the specification must commence on a separate sheet, preferably following the claims, under the heading “Abstract of the Disclosure.” The purpose of the abstract is to enable the Patent and Trademark Office and the public generally to determine quickly from a cursory inspection the nature and gist of the technical disclosure. The abstract shall not be used for interpreting the scope of the claims.

Therefore, any statements made relating to the abstract are not intended to limit the claims in any manner and should not be interpreted as limiting the claims in any manner.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

What is claimed is:

1. A beverage bottling plant for filling beverage bottles with liquid beverage material, said beverage bottling plant comprising:
a beverage bottle cleaning machine being configured and disposed to clean beverage bottles;
a feed arrangement to supply beverage bottles to said beverage bottle cleaning machine;
a beverage filling machine being configured and disposed to fill beverage bottles with liquid beverage material;
said beverage filling machine comprising a plurality of beverage filling elements for filling beverage bottles with liquid beverage material;
at least one liquid reservoir being configured to hold a liquid to be bottled;
said at least one liquid reservoir comprising a gas headspace being disposed above a liquid to be bottled within said at least one liquid reservoir;
at least one supply line being configured and disposed to connect said at least one liquid reservoir to said beverage filling machine to supply liquid beverage material to said beverage filling machine;
a first conveyer arrangement being configured and disposed to move beverage bottles from said beverage bottle cleaning machine into said beverage filling machine;
said first conveyer arrangement comprising a star wheel structure;
a beverage bottle closing machine being configured and disposed to close tops of filled beverage bottles;
a second conveyer arrangement being configured and disposed to move filled beverage bottles from said beverage filling machine into said beverage bottle closing machine;
said second conveyer arrangement comprising a star wheel structure;
a beverage bottle labeling machine being configured and disposed to label filled, closed beverage bottles;
a third conveyor arrangement being configured and disposed to move filled, closed beverage bottles from said beverage bottle closing machine into said beverage bottle labeling machine;
said third conveyer arrangement comprising a star wheel structure;
a beverage bottle packing station being configured and disposed to package labeled, filled, closed beverage bottles;
a fourth conveyor arrangement being configured and disposed to move labeled, filled, closed beverage bottles from said beverage bottle labeling machine to said beverage bottle packing station;
said fourth conveyer arrangement comprising a linear conveyor structure being configured and disposed to arrange beverage bottles in groups for packing;
a computer control system being configured and disposed to monitor and control operation of said beverage bottling plant;
a beverage bottle treatment machine being configured and disposed to treat bottles to be filled;
said beverage bottle treatment machine comprising:
a treatment device being configured and disposed to treat the insides of bottles to be filled, comprising:
a supply for containing treatment agent to treat bottles;
a spray device being configured and disposed to introduce compressed air into said treatment device and to mix said compressed air with a supply of treatment agent to create treated air to treat bottles;
a heater or heat exchanger being configured and disposed to heat treated air;
a helical heating channel being configured and disposed to heat treated air and to permit the flow of treated air;
a vertical tube being configured and disposed to permit the flow of treated air into a bottle;
said tube comprising a dispensing opening being configured and disposed to permit the flow of treated air into a bottle;
said helical heating channel being disposed about said tube;
a housing being configured and disposed to house said heater or heat exchanger, said tube, and said helical heating channel;
an enhancer being configured and disposed to enhance treated air prior to the exit of treated air from said tube.

2. Container treatment machine according to claim 1, wherein:
the at least one enhancer is located on the path of the treatment agent from its source to the container to be treated;
the at least one enhancer is located inside a tube that conducts the treatment agent and/or its decomposition products into or to the container;
the enhancer is also realized in the form of a tube that conducts the treatment agent and/or its decomposition products in or to the container and is made of a porous material;
the surface of the heat exchanger is at least partly realized in the form of an enhancer;
the heat exchanger is made at least partly of catalytically active material;
the at least one enhancer is made of noble metals of the platinum group and/or magnesium dioxide or brownstone;
the at least one enhancer is manufactured by means of powder-metallurgical processes;
the at least one enhancer comprises wire meshes manufactured from small-diameter wire;
the at least one enhancer is realized in the form of a heating element for the treatment agent and/or its decomposition products;
at least one enhancer is associated directly with each treatment device;
at least one enhancer is directly associated with a group of treatment devices; and
at least one enhancer is directly associated with all the treatment devices of a container treatment machine.

3. A method of treating bottles with a treatment device, said treatment device comprising: a supply for containing treatment agent to treat bottles; a spray device being configured and disposed to introduce compressed air into said treatment device and to mix said compressed air with a supply of treatment agent to create treated air to treat bottles; a heater or heat exchanger being configured and disposed to heat treated air; a helical heating channel being configured and disposed to heat treated air and to permit the flow of treated air; a vertical tube being configured and disposed to permit the flow of treated air into a bottle; said tube comprising a dispensing opening being configured and disposed to permit the flow of treated air into a bottle; said helical heating channel being disposed about said tube; a housing being configured and disposed to house said heater or heat exchanger, said tube, and said helical heating channel; an enhancer being configured and disposed to enhance treated air prior to the exit of treated air from said tube; said method comprising the steps of:
mixing treatment agent and compressed air to create treated air;
heating treated air with said heater or heat exchanger;
flowing air through said enhancer to enhance treated air;
enhancing treated air at a temperature below the maximum allowable temperature of the containers to be treated;
flowing enhanced, treated air into a container to be treated.

4. The method according to claim 3, wherein:
the at least one enhancer is located on the path of the treatment agent from its source to the container to be treated;
the at least one enhancer is located inside a tube that conducts the treatment agent and/or its decomposition products into or to the container;
the enhancer is also realized in the form of a tube that conducts the treatment agent and/or its decomposition products in or to the container and is made of a porous material;
the surface of the heat exchanger is at least partly realized in the form of an enhancer;
the heat exchanger is made at least partly of catalytically active material;
the at least one enhancer is made of noble metals of the platinum group and/or magnesium dioxide or brownstone;
the at least one enhancer is manufactured by means of powder-metallurgical processes;
the at least one enhancer comprises wire meshes manufactured from small-diameter wire;
the at least one enhancer is realized in the form of a heating element for the treatment agent and/or its decomposition products;
at least one enhancer is associated directly with each treatment device;
at least one enhancer is directly associated with a group of treatment devices; and
at least one enhancer is directly associated with all the treatment devices of a container treatment machine.

5. The method according to claim 4, wherein:
the decomposition of the treatment agent takes place at a temperature below the shape recovery and/or contraction temperature of plastic containers; and
the decomposition of the treatment agent occurs at room temperature, preferably in the temperature range of 15 to 25° C.

6. A container treatment device being configured and disposed to treat the insides of containers to be filled, comprising:
a supply for containing treatment agent to treat containers;
a spray device being configured and disposed to introduce compressed gas into said treatment device and to mix said compressed gas with a supply of treatment agent to create a treated gas to treat containers;
a heater or heat exchanger being configured and disposed to heat treated gas;
a tube being configured and disposed to permit the flow of treated gas into a container;
said tube comprising a dispensing opening being configured and disposed to permit the flow of treated gas into a container;
an enhancer being configured and disposed to enhance treated gas exiting from said tube.

7. Container treatment device according to claim 6, wherein the at least one enhancer is located in the path of the treatment agent, which is hydrogen peroxide, from its source to the container to be treated.

8. Container treatment device according to claim 7, wherein the at least one enhancer is located inside a tube that conducts the treatment agent and/or its decomposition products into or to the container.

9. Container treatment device according to claim 8, wherein the enhancer is also realized in the form of a tube that conducts the treatment agent and/or its decomposition products in or to the container and is made of a porous material.

10. Container treatment device according to claim 9, wherein the surface of the heat exchanger is at least partly realized in the form of an enhancer.

11. Container treatment device according to claim 10, wherein the heat exchanger is made at least partly of catalytically active material.

12. Container treatment device according to claim 11, wherein the at least one enhancer is made of noble metals of the platinum group and/or magnesium dioxide or brownstone.

13. Container treatment device according to claim 12, wherein the at least one enhancer is manufactured by means of powder-metallurgical processes.

14. Container treatment device according to claim 13, wherein the at least one enhancer comprises wire meshes manufactured from small-diameter wire.

15. Container treatment device according to claim 14, wherein the at least one enhancer is realized in the form of a heating element for the treatment agent and/or its decomposition products.

16. Container treatment device according to claim 15, wherein at least one enhancer is associated directly with each treatment device.

17. Container treatment device according to claim 16, wherein at least one enhancer is directly associated with a group of treatment devices.

18. Container treatment device according to claim 17, wherein at least one enhancer is directly associated with all the treatment devices of a container treatment machine.

19. Container treatment device according to claim 18 wherein the method for the operation of said container treatment device is, characterized by the fact that the decomposition of the treatment agent takes place at a temperature below the shape recovery and/or contraction temperature of plastic containers.

20. Container treatment device according to claim 19 wherein the method for the operation of a container treatment device is characterized by the fact that the decomposition of the treatment agent occurs at room temperature, preferably in the temperature range of fifteen degrees Celcius to twenty-five degrees Celcius.
